(19) United States
(12) Patent Application Publication
(10) Pub. No.: US 2025/0241764 A1
(43) Pub. Date: Jul. 31, 2025

(54) SYSTEMS AND METHODS FOR PROVIDING AND DEPLOYING INTERBODY IMPLANTS

(71) Applicant: Nexus Spine, LLC, Salt Lake City, UT (US)

(72) Inventors: Peter A. Halverson, Salt Lake City, UT (US); David T. Hawkes, Salt Lake City, UT (US)

(21) Appl. No.: 19/046,436

(22) Filed: Feb. 5, 2025

Related U.S. Application Data
(63) Continuation-in-part of application No. 18/623,983, filed on Apr. 1, 2024.
Application No. 18/623,983 is a continuation of application No. 16/297,290, filed on Mar. 8, 2019, now Pat. No. 11,944,552.
(60) Provisional application No. 62/640,556, filed on Mar. 8, 2018.
(60) Provisional application No. 62/689,703, filed on Jun. 25, 2018.
(60) Provisional application No. 62/689,707, filed on Jun. 25, 2018.
(60) Provisional application No. 63/550,491, filed on Feb. 6, 2024.

(51) Int. Cl. A61F 2/44; A61F 2/30; A61F 2/46

(52) U.S. Cl. CPC A61F 2/4455; A61F 2/30749; A61F 2/442; A61F 2/4611; A61F 2002/3028; A61F 2002/30579; A61F 2002/30593; A61F 2002/30622; A61F 2002/30845; A61F 2002/30892

(57) ABSTRACT

Systems and methods for providing an interbody implant are disclosed. According to some implementations, the implant includes an implant body having a cranial face and a caudal face disposed opposite the cranial face. In some implementations, the implant includes one or more primary fixation mechanisms, which in turn can include one or more anchor cavities defined by the implant body. In some cases, one or more anchors are configured to be housed at least partially within the anchor cavities in an undeployed position, and the anchors are configured to be translationally deployed to a deployed position in order to couple the implant to a bony endplate of a spinal vertebra. In some implementations, the implant includes one or more actuators configured to effectuate selective deployment of the anchors.

### RELATED APPLICATIONS

This is a continuation-in-part of U.S. patent application Ser. No. 18/623,983 (Attorney Docket No. 23845.179), entitled Stand-Alone Interbody Fusion, filed Apr. 1, 2024, which itself is a continuation of U.S. patent application Ser. No. 16/297,290 (Attorney Docket No. 23845.97; now U.S. Pat. No. 11,944,552), entitled Stand-Alone Interbody Fusion, and filed Mar. 8, 2019, which claims the benefit of: U.S. Provisional Application No. 62/640,556 (Attorney Docket No. 23845.89), entitled Stand-Alone Interbody Fusion, and filed Mar. 8, 2018; U.S. Provisional Application No. 62/689,703 (Attorney Docket No. 23845.96), entitled Stable-C Standalone Interbody, and filed Jun. 25, 2018; and U.S. Provisional Application No. 62/689,707 (Attorney Docket No. 23845.95), entitled Interbody Inserter, and filed Jun. 25, 2018. This application also claims priority to U.S. Provisional Patent Application No. 63/550,491, entitled Systems and Methods for Providing and Deploying Interbody Implants (Attorney Docket No. 23845.177), filed Feb. 6, 2024. Each and every one of the foregoing applications is incorporated herein by reference, in their entireties, and for all they disclose.

### FIELD

The present disclosure relates to medical devices, and more particularly to interbody spinal implants and methods for deploying such implants and providing primary fixation for the same.

### BACKGROUND

Traditionally, some interbody implants are anchored to the vertebra of one or more spinal segments to provide stability and to prevent the implants from shifting out of place, which could potentially cause pain, or even damage to the patient. However, many traditional interbody implants have limitations. For example, some implants have anchors that: are cumbersome to deploy, require fiddly instruments, are bulky, or must be forced into the bony endplate in a manner that tends to fracture or otherwise damage the vertebra. Additionally, some implants do not provide sufficient stability, notwithstanding the presence of anchors.

Thus, while techniques currently exist that are used to provide and deploy an interbody implant, challenges still exist, including those listed above. Accordingly, it would be an improvement in the art to augment or even replace current techniques with other techniques.

### SUMMARY

Systems and methods for providing spinal interbody spacer implants (or implants) are provided herein, particularly implants having improved primary fixation characteristics. The described implant can include any suitable components or features that allow it to perform its functions as an interbody spacer (e.g., to provide a fixed space between spinal vertebrae, restore disc height, reduce nerve compression, eliminate or reduce pain, promote bone growth, or otherwise treat spinal conditions). Accordingly, the implant can include any suitable features of an interbody spacer. For example, some implementations include one or more implant bodies, which may have one or more faces (or surfaces), such as a cranial face, a caudal face, a ventral face, a dorsal face, a first lateral face (e.g., a left face), a second lateral face (e.g., a right face), and any other suitable face.

Some implementations include one or more inserter interfaces for coupling to or otherwise interfacing with an insertion tool (to simplify the insertion process). Some iterations include one or more frames, which can include any components for providing a particular shape or structure for the implant. In some cases, the implant includes one or more passages, such as one or more cavities configured to be filled with biocompatible materials for promoting bone grown. In some implementations, the implant includes one or more porous structures configured to optimize bone growth, provide a desirable compression stiffness, or provide the implant with other desirable characteristics. The implant can also have any other suitable component or attribute of an interbody spacer implant, as discussed in additional detail below or as known in the art.

To provide improved primary fixation characteristics, some implementations of the implant include one or more dedicated primary fixation mechanisms. The primary fixation mechanism can include any suitable component or feature as may be useful for coupling or affixing the implant to one or more of the patient's bony endplates adjacent to the implant when the implant is installed in its proper place.

According to some implementations, the primary fixation mechanism includes one or more actuators configured to deploy the anchor (e.g., to move the anchor from an undeployed position to a deployed position). The actuator can include any automatic, semi-automatic, mechanical, or manual mechanism for moving the anchor. That said, some iterations of the actuator are configured to move the anchor translationally (e.g., linearly) such that the anchor can move straight up cranially into a bony endplate above the implant, or straight down caudally into a bony endplate below the implant.

In some implementations, the actuator includes one or more cams configured to convert rotational motion to translational motion. For example, in some embodiments, when the cam or a portion thereof is rotated, the cam pushes the anchor linearly, causing the anchor to deploy without changing its orientation. In some cases, this can lead to an anchor deployment that causes minimal damage to the patient's bony endplate, and that also places the anchor in an optimal position to resist movement of the implant back out through the opposite path and direction of insertion (e.g., as the anchor may be positioned orthogonally to the direction of insertion).

Although the cam can operate in any suitable manner, some instances of the cam include one or more lobes coupled to one or more shafts. In some cases, the shaft is disposed near a perimeter of the lobe, such that it moves cranially or caudally as the lobe rotates. Some implementations of the anchor are configured to be pushed by the shaft, thereby moving translationally in response to rotation of the lobe. Furthermore, some instances of the lobe include one or more engagement features configured to engage with one or more keys that can be used to rotate the lobe. Thus, in some cases, rotation (and thereby translational deployment of the anchor) can be accomplished easily by simply rotating the key (or any other suitable component) while it is engaged with the lobe.

In some cases, the anchor includes one or more cutouts configured to optimize interaction between the anchor and the shaft of the cam. For example, the cutout can have contours that smoothen the motion of the anchor, ensure even application of the pressure of the shaft on the anchor, prevent retrograde (backward) movement of the cam, or otherwise assist in providing desirable characteristics for the anchor deployment.

Some implementations of the primary fixation mechanism include one or more limiters configured to limit undesirable motion of the primary fixation mechanism. For example, in some iterations, the limiters are configured to prevent or otherwise limit undesirable retraction of the anchor (in some cases, selectively, such that the anchor can still be retracted if desired), or to limit rotation of the cam to a single direction (e.g., clockwise or counterclockwise, as desirable).

The primary fixation mechanism, and the implant in general, can have many other features to assist in improved anchor deployment or otherwise improve the functionality of the implant. Accordingly, the features described in this summary, along with other useful features, are discussed in greater detail below.

### BRIEF DESCRIPTION OF FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1 shows a front elevation view of an interbody implant, in accordance with some representative embodiments of the disclosed systems and methods;

FIG. 2 shows a side elevation view of the interbody implant, in accordance with some representative embodiments;

FIG. 3 shows a front perspective view of the interbody implant, in accordance with some representative embodiments;

FIG. 4A shows a front elevation view of the interbody implant, in accordance with some representative embodiments;

FIG. 4B shows a front elevation view of the interbody implant with deployed anchors, in accordance with some representative embodiments;

FIG. 5 shows a front X-ray view of the interbody implant with a focus on a primary fixation mechanism of the interbody implant, in accordance with some representative embodiments;

FIG. 6A shows a front perspective view of the primary fixation mechanism of the interbody implant, in accordance with some representative embodiments;

FIG. 6B shows a front elevation view of the primary fixation mechanism of the interbody implant, in accordance with some representative embodiments;

FIG. 6C shows a front perspective view of a cam of the primary fixation mechanism of the interbody implant, in accordance with some representative embodiments;

FIG. 6D shows a front elevation view of an anchor of the primary fixation mechanism of the interbody implant, in accordance with some representative embodiments;

FIGS. 7A-7C show front perspective views of the interbody implant, illustrating how a key can be used to deploy anchors thereof, in accordance with the representative embodiments;

FIGS. 8A-8B show rear X-ray views of the interbody implant, with a focus on a dorsal side of the primary fixation mechanism, in accordance with some representative embodiments;

FIG. 9A shows a front perspective view of the implant having an inserter interface in the form of a first window and a second window, in accordance with some embodiments;

FIG. 9B shows a front elevation view of the implant having an inserter interface in the form of a first window and a second window, in accordance with some embodiments;

FIG. 9C shows a plan view of the implant, in accordance with some embodiments; and

FIGS. 9D-9E show cross-sectional front elevation views of the implant through cross section A-A of FIG. 9C, with FIG. 9D showing a first cam position and FIG. 9E showing a second cam position, in accordance with some embodiments.

### DETAILED DESCRIPTION

A description of embodiments will now be given. It is expected that the present systems and methods may take many other forms and shapes. Accordingly, the following disclosure is intended to be illustrative and not limiting.

Generally speaking, interbody implants (or implants) are configured to be inserted between two vertebrae of a spine (e.g., a human spine). Inserting an implant often requires resection of tissue from the space into which the implant is to be inserted. Furthermore, after an implant is inserted, it has a tendency to move out of place (e.g., natural movement of the human body can result in bumps, jostles, vibrations, and other motions that can cause the implant to shift from its target location). In particular, implants are frequently inclined to shift out of place in a direction opposite to a direction of insertion. For example, if an implant is inserted in a ventral-to-dorsal direction (front to back), the implant often has a tendency to shift out of place in a dorsal-to-ventral direction (back to front). If an implant is inserted laterally, the implant often has a tendency to shift out of place laterally (i.e., back the way it came). Thus, implants often have a tendency to move in a direction parallel to a direction of insertion.

To resist an implant's natural tendency to shift out of place, some implants have an anchoring mechanism, which can include spikes, blades, or other inserts configured to insert into a bony endplate and anchor the implant in place. That said, deployment of such inserts often causes damage to the patient's spine. For example, many conventional inserts are configured to be hammered into place, which can crack bone, cause vibrational trauma, and otherwise harm a patient. Many other inserts are configured to be rotated into place, which can cut gouges in the bony endplates of the patient's vertebral bodies. Moreover, many inserts are generally aligned with the direction of insertion, which means that they can resist motion orthogonal to the direction of insertion more than motion parallel to the direction of insertion (when parallel motion is more likely to occur, as discussed above). Accordingly, some such anchors can cause further damage to the patient's spine, cutting gouges or even pulling free of the bony endplate as they migrate in a direction parallel to their blades.

Furthermore, some implants require many inserts or overly large inserts to retain the implant in place, which can cause additional damage, and consequently pain and degradation of spinal integrity, to the patient. Moreover, in the case of spinal degeneration, bony endplates may become thickened (e.g., become sclerotic) or otherwise altered, thereby increasing the amount of force required to penetrate the bony endplates. Thus, some implants have difficulties that arise as a result of spinal degeneration, such as having anchors that buckle, skive, or otherwise deflect in an undesirable direction.

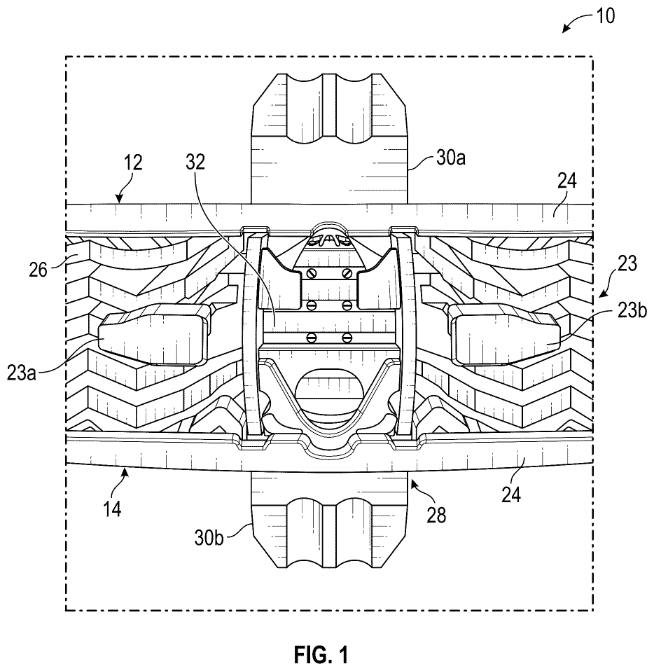

Embodiments of the systems and methods disclosed herein address these and other shortcomings. Indeed, according to some embodiments, one or more spinal interbody spacer implants (implants) 10 are provided. The implant can have any suitable component or configuration that allows it to operate as an interbody spacer implant. For example, some embodiments include an implant body having one or more: cranial surfaces 12; caudal surfaces 14; ventral surfaces 16; dorsal surfaces 18; first lateral surfaces 20 (e.g., a left or right surface); second lateral surfaces 22 (e.g., another left or right surface), or any other suitable surfaces. The implant can (as shown in FIGS. 1-9E) also include one or more inserter interfaces 23, frames 24, passages 25, porous structures 26, primary fixation mechanisms 28, or any other suitable component. Indeed, other features can also be included, including as described in additional detail below.

Where the implant 10 includes one or more of a cranial surface 12, caudal surface 14, ventral surface 16, dorsal surface 18, first or second lateral surfaces 20, 22, or any other suitable surfaces, any of the surfaces can have any suitable feature, such as being generally straight. For example, in some cases, a surface is highly straight (being straight or within 1 degree of straight, or having less than a 1 degree deflection, curve, or other deviation from straight), substantially straight (e.g., being straight or within 3 degrees of straight, or having less than a 3 degree deflection, curve, or other deviation from straight), largely straight (being straight or within 10 degrees of straight, or having less than a 10 degree deflection, curve, or other deviation from straight). In some embodiments, one or more surfaces is curved, wavy, undulated, solid, porous, texturized, smooth, comprises raised portions, comprises recessed portions, or is otherwise configured. In some cases, the various surfaces are clearly defined, such that the implant is generally formed in a cuboid shape, whereas in some cases one or more of the surfaces are curved to such a degree that they run together (for example, in some cases, the lateral surfaces, the ventral surface, and the dorsal surface are all part of a generally circular or oval surface that extends around a perimeter of the implant). Thus, in some cases, the implant is generally cylindrical. The implant body can also have another generally polyhedral configuration, or any other general shape suitable for use as an interbody spacer.

In some embodiments, the cranial surface 12 is disposed opposite the caudal surface 14, and these surfaces are configured to be disposed adjacent to the patient's bony endplates of adjacent vertebral bodies. For example, the cranial surface is configured to be disposed adjacent to a bony endplate of a vertebra cranial to (e.g., above) the implant 10, and the caudal surface is configured to be disposed adjacent to a bony endplate of a vertebra caudal to (e.g., below) the implant. In some cases, the cranial surface and the caudal surface are generally parallel, such as highly parallel (e.g., parallel or within 1 degree of parallel), substantially parallel (e.g., parallel or within 3 degrees of parallel), or largely parallel (e.g., parallel or within 10 degrees of parallel) to each other (or even, in some cases, simply more parallel than perpendicular to each other). That said, in some cases, the cranial surface and the caudal surface are angled with respect to each other (in any direction, such that the dorsal surface is shorter than the ventral surface (or vice versa) or one lateral surface is shorter than the other, or some combination of the foregoing). In such cases, the angle can be any suitable angle, such as between 0 degrees and 45 degrees, or any subrange thereof (e.g., between 0 degrees and: 1 degree, 2 degrees, 3 degrees, 5 degrees, 10 degrees, 20 degrees, 30 degrees, or any other suitable angle). Similarly, the first lateral surface 20 is (in some embodiments) disposed opposite the second lateral surface 22 (in some cases, the lateral surfaces are highly parallel, substantially parallel, or largely parallel to each other, and in some cases, they are angled with respect to each other as discussed above). In some embodiments, the ventral surface 16 is disposed opposite the dorsal surface 18 (in some cases, the ventral surface and the dorsal surface are highly parallel, substantially parallel, or largely parallel to each other, and in some cases, they are angled with respect to each other as discussed above).

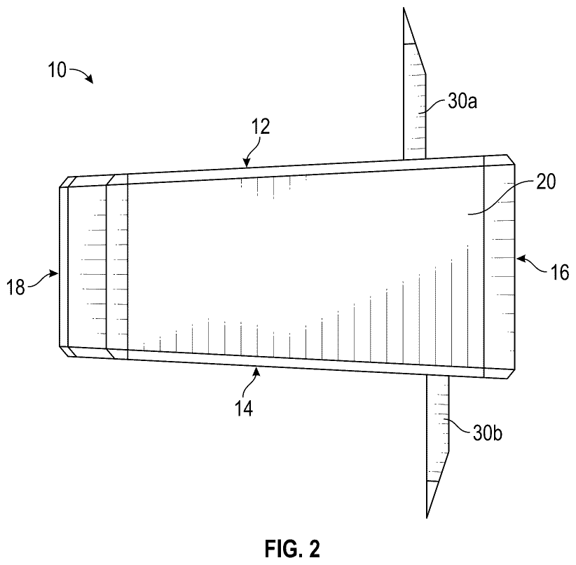

By way of non-limiting illustration, FIG. 1 shows an implant 10 having a cranial surface 12 disposed opposite a caudal surface 14. By way of further illustration, FIGS. 2-3 show implants 10, each having a general cuboid shape, and each having a cranial surface 12, a caudal surface 14, a ventral surface 16, a dorsal surface 18, a first lateral surface 20, and a second lateral surface 22. In the embodiments shown in FIGS. 2-3, the various surfaces are substantially flat (although in some cases there are beveled or rounded edges where the surfaces join together). Furthermore, FIG. 2 shows an example of an embodiment in which the cranial surface 12 and the caudal surface 14 are angled with respect to each other.

The implant 10 can have any suitable size. Generally speaking, the size of the implant is whatever size is needed to effectively perform the function of an interbody implant. In some cases, different sizes of implants are used for different spinal locations (for cervical, thoracic, and lumbar vertebrae) and for patients of different sizes or with any other different characteristics (e.g., weight, bone size, bone shape, bone density, etc.).

According to some embodiments, the implant 10 includes one or more inserter interfaces 23. Where the implant includes an inserter interface, the inserter interface can include any component configured to interface with an insertion tool. For example, the inserter interface can include one or more tabs, stops, catches, hooks, protrusions, indentations, openings, windows, grooves, notches, slots, processes, recesses, keys, keyholes, or any other suitable components or features configured to allow the implant to (selectively) couple to or be moved by an insertion tool. For example, some embodiments are configured to interface with an insertion tool as disclosed in U.S. patent application Ser. No. 16/452,512, with the title INTERBODY INSERTER, filed Jun. 25, 2019, which is incorporated herein by reference in its entirety.

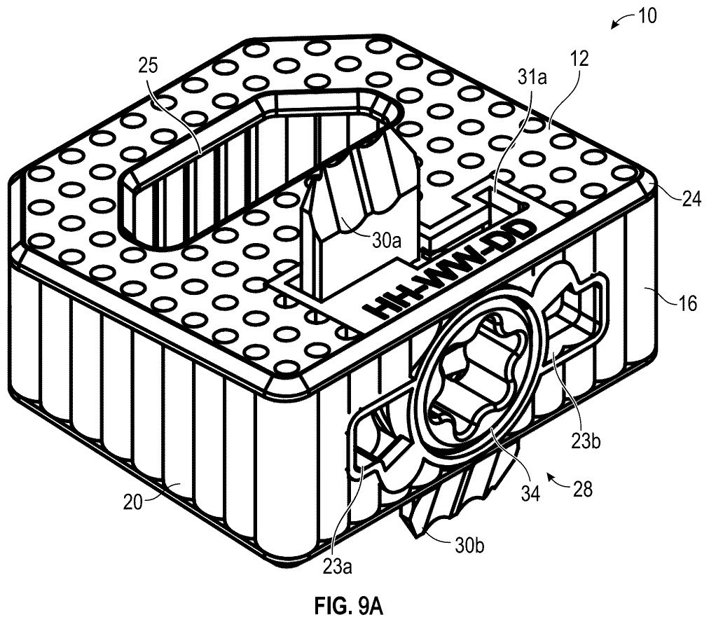

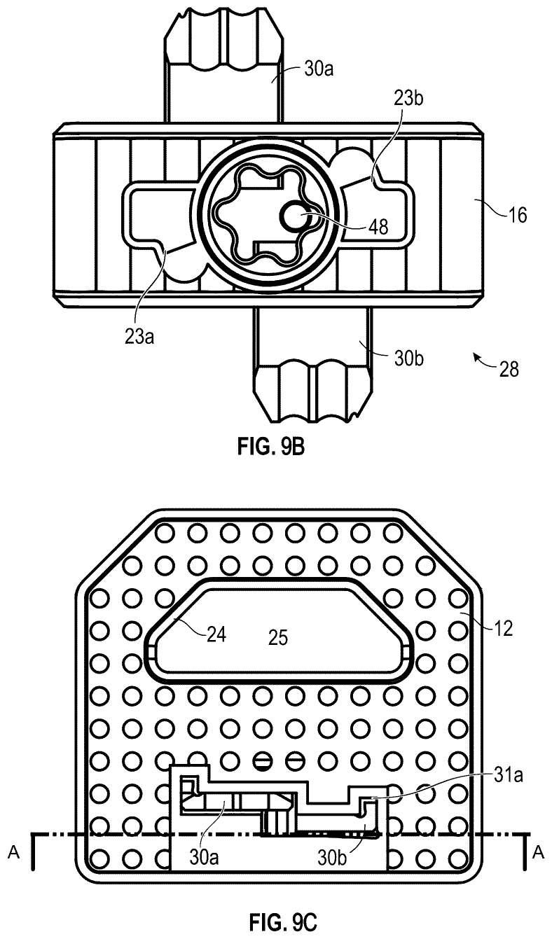

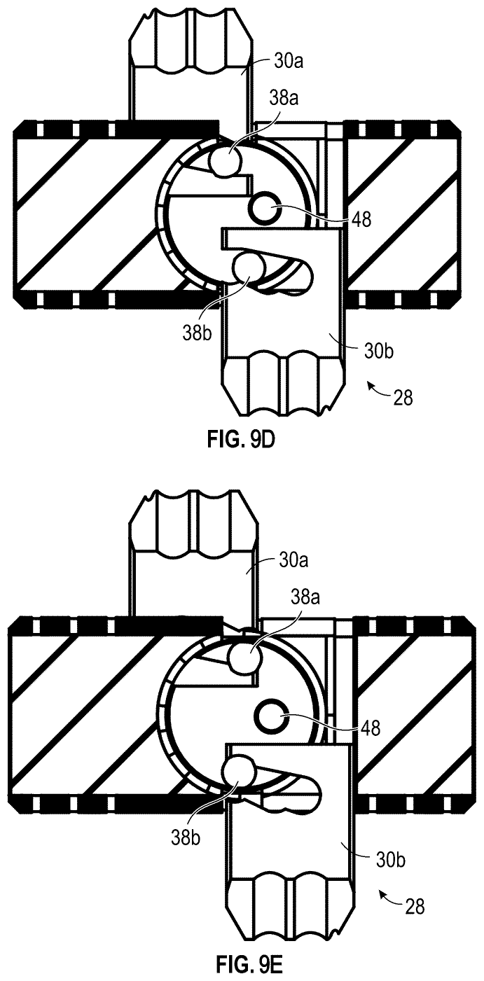

According to some embodiments, the inserter interface 23 includes a first catch 23a and a second catch 23b, with each catch being configured to engage a component of an insertion tool (e.g., a flexible tab, a corresponding catch, or another engagement component). According to some embodiments, the first catch and the second catch are disposed on (or formed in) the ventral surface 16 (though the first or second catch are, in some embodiments, disposed at any other suitable surface, such as at the first lateral 20 or second lateral 22 surface). In some embodiments, the catches are disposed on opposite sides of a primary fixation mechanism 28 (such as any iteration of a primary fixation mechanism as described in additional detail below). Indeed, some embodiments of the catches are spaced apart from a primary fixation mechanism such that the primary fixation mechanism can be actuated without interference from the inserter interface. Although the first catch and the second catch can be joined together (and may be integrally formed with the implant 10), in some cases, the first catch and the second catch are separate components that are separately coupled to a body of the implant. Some iterations of the first catch and the second catch are configured to be flush with the applicable surface (e.g., the ventral surface 16, or another applicable surface), such that they do not protrude beyond such surface. For example, some embodiments of the catches are recessed into the applicable surface, and some embodiments of the catches are substantially in the form of windows or other non-protruding components.

Where the first catch 23a and the second catch 23b include one or more windows, slots, indentations, depressions, or other passages, the windows can have any suitable shape (e.g., as may be useful for engaging with an insertion tool). By way of non-limiting illustration, FIGS. 9A-9B show an implant 10 having an inserter interface with a first catch 23a and a second catch 23b that include windows, the windows having a substantially rectangular shape with an elongated portion formed along a part of one side thereof.

In some cases, the inserter interface 23 is coupled to a frame 24 of the implant (as discussed in additional detail below), but in some cases the inserter interface is separate from the frame (e.g., the inserter interface is not directly coupled to the frame, but rather it is coupled to or mounted on (or formed in) a body of the implant.

In some cases, the insertion tool is configured not to inadvertently disengage from the implant 10 (e.g., if the tool is tilted or rotated, such as due to pressures exerted on the implant from movement of the spine, or for any other reason). Furthermore, in some embodiments, the insertion tool is provided along with the implant as part of an interbody implant system.

According to some embodiments, the implant 10 includes one or more frames 24. Where the implant includes a frame, the frame can include any suitable component configured to provide structure or shape to the implant or to impart on the implant (or modify) any suitable characteristic, such as stiffness, size, shape, strength, or any other suitable characteristic. In some cases, the frame includes one or more bands, rings, discs, or other generally annular elements (whether circular, rectangular, square, polygonal, or otherwise), but the frame can also include one or more elements of any other shape (e.g., semi-circular, triangular, square, rectangular, trapezoidal, pentagonal, hexagonal, star-shaped, T-shaped, polygonal, symmetrical, asymmetrical, or any other regular or irregular shape). In some cases, the frame includes one or more structural elements extending along a perimeter of (in some cases, surrounding the perimeter, either in a contiguous or broken manner) the cranial surface 12 or the caudal surface 14 (or the ventral surface 16, the dorsal surface 18, the first lateral surface 20, the second lateral surface 22, or any other surface). According to some embodiments, the frame at least partially covers one or more portions of one or more of the surfaces (e.g., a perimeter portion, a ventral portion, a dorsal portion, a lateral portion, a middle portion, or any other portion). That said, in some embodiments, the frame is configured to leave one or more particular portions of the implant exposed (e.g., by not covering or extending across them). For example, in some cases, the frame is configured not to cover or extend across the cranial surface, the caudal surface, the ventral surface, the dorsal surface, the first lateral surface, the second lateral surface, or any particular portion of any such surfaces (e.g., such surfaces or portions of such surfaces are free of frame components). In fact, some embodiments of the implant are free of a frame altogether. In some other embodiments, a frame is included, but the frame is low-profile, low volume, or otherwise configured to avoid being overly bulky or interfering with other components or features of the implant. For example, in some cases, the frame takes up less than 25% (or any other suitable percent less than 25%, such as 15%, 10%, 5%, or 1%) of the implant by at least one of volume and mass.

According to some embodiments, the implant 10 includes one or more passages. The passage can include any suitable cavities, receptacles, slots, recesses, voids, notches, grooves, holes, bores, depressions, orifices, or other passages. In some embodiments, the passage is free of the porous structure 26 (or other material forming a body of the implant). In some cases, the passage is defined by the body of the implant (e.g., it is formed therein). The passage can be formed in or through any portion of the implant, such as in a single face (e.g., in the ventral surface, the cranial surface, the caudal surface, etc.), from any one face to another (e.g., from the cranial surface to the caudal surface), internally (e.g., without having an opening in any face), or in any other suitable manner. The passage can also have any suitable dimensions, such as by accounting for anywhere between 1% and 50% of the volume of the implant (or any subrange thereof, such as 10%±5%, 15%±7%, 25%±12%, or any other subrange of the implant's volume, as defined by the external most perimeters of the implant).

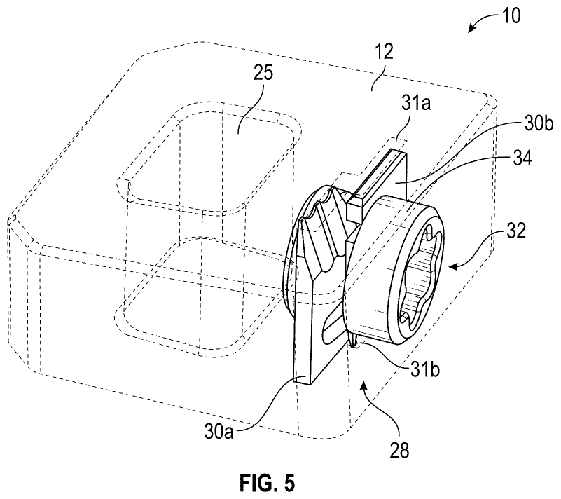

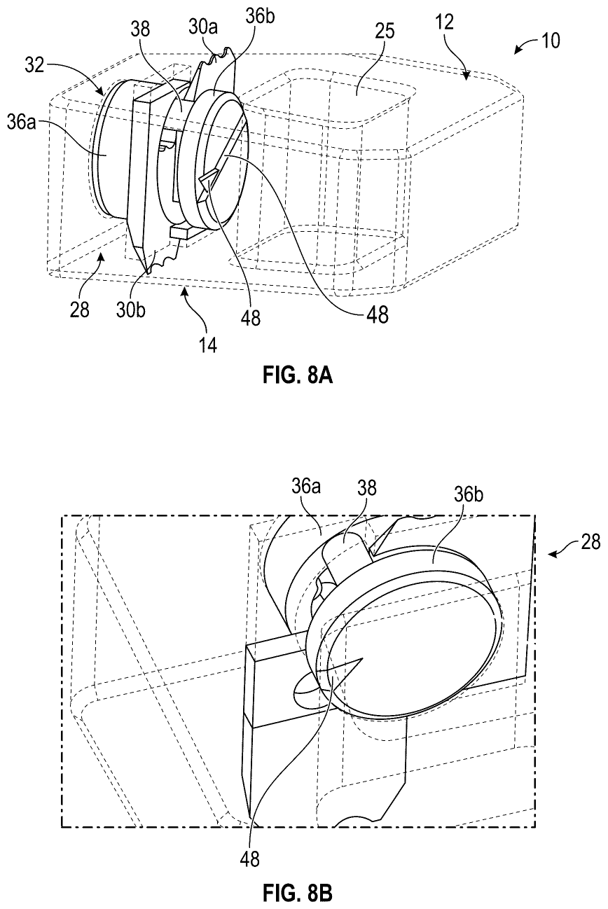

According to some embodiments having a passage, the passage includes one or more cavities 25. While such a cavity can perform any suitable function, in some embodiments it is configured to receive one or more bone graft materials, such as morselized bone (living bone), thereby providing a seed for initiating bone growth into the implant 10 after implantation. In some embodiments, the cavity extends only partway through the implant, but some embodiments of the cavity extend from one surface of the implant to another (e.g., from the cranial surface to the caudal surface). The cavity can also be positioned on any portion of the implant, at any angle, or with any other suitable configuration as may be useful for providing bone graft materials. The cavity can further have any suitable shape for receiving bone graft materials. By way of non-limiting illustration, FIGS. 5 and 8A show implants 10 having a cavity 25 formed from the cranial surface 12 to the caudal surface 14, extending through an entirety of the implant, generally centered between the first lateral surface 20 and the second lateral surface 22, and slightly off-center dorsally between the ventral surface 16 and the dorsal surface 18. Notwithstanding the foregoing, some embodiments do not have a central cavity (e.g., the porous structure or body of the implant extends through the center of the implant or the center of any of the surfaces thereof).

According to some embodiments having a passage, the passage includes one or more elements of a primary fixation mechanism 28, such as one or more anchor cavities 31 (as discussed in more detail below).

According to some embodiments, a body of the implant 10 includes one or more porous structures 26. In this regard, a properly configured porous structure can be highly beneficial in promoting healthy bone growth. Indeed, the cells generally responsible for bone growth, namely osteocytes and osteoblasts, typically work together to form bone as needed within the body. But these cells will typically only form bone under proper conditions, including when the cells experience proper loads and stresses, when a network of blood vessels is available to supply needed nutrients, and when gaps to be filled by bone are of a proper size. When proper conditions are not available, bone often cannot or will not grow. For example, when bone does not experience proper loading (e.g., due to hypoloading), it generally will not grow and can even be resorbed. Additionally, when gaps (i.e., pores) to be filled are too large, bone may not be able to bridge the gap and often will not grow. Similarly, if pores are too small, bone growth may be impeded by the insufficient size. Thus, some embodiments of the implant employ a combination of a porous structure with proper loading (e.g., as a result of the conforming region) to stimulate improved bone growth.

Where embodiments include a porous structure 26, the porous structure can include any structure having gaps, spaces, air pockets, voids, cavities, hollows, open cells, closed cells, recesses, or other pores capable of promoting bone growth (e.g., cortical bone growth, trabecular bone growth, or other bone growth).

According to some embodiments, the porous structure 26 includes one or more pores, or one or more pluralities of pores. The pores can include any suitable gaps, spaces, voids, air pockets, cavities, hollows, open cells, closed cells, recesses, or other pores that are defined by the porous structure. For example, a hole in a strand qualifies as a pore (in some embodiments), and a gap between multiple strands (or multiple portions of the same strand) qualifies as a pore (in some embodiments).

In some embodiments, each pore of the plurality of pores has a pore size, or the plurality of pores has an average pore size, of between 10 μm and 3,000 μm or any subrange thereof. For example, in some embodiments, the plurality of pores each have a pore size, or they have an average pore size, of between 100 μm and 1,000 μm, between 150 μm and 700 μm, between 150 μm and 650 μm, or any other subrange of between 10 μm and 3,000 μm. That said, a pore size of between approximately 150 μm and 650 μm (±100 μm) can (in accordance with some embodiments) be particularly useful for promoting bone growth in combination with the loads placed on bony endplates 20 as a result of the stiffnesses selected for the load-bearing regions of some embodiments of the implant 100 (as discussed above).

The plurality of pores can include any number of pores (e.g., between 2 and 500,000, or any subrange thereof, such as 100±50, 1000±500, 1500±750, 10,000±5000, or any other desired number of pores for promoting bone growth). The pores can also be positioned in any manner that can be helpful for promoting bone growth, but in some embodiments, they are at least partially exposed at one or more faces of the implant 100 (e.g., any or all of the caudal face, the cranial face, the ventral face, the dorsal face, the left face, the right face, or any other suitable surface), which in some cases allows bone to access (e.g., grow into) the pores or allows for fluid to circulate within the implant.

In some embodiments, two or more of the plurality of pores are interconnected (e.g., each of the pores of the plurality of pores is fluidically interconnected with one or more of the other pores of the plurality of pores). In some embodiments, the porous structure 26 includes multiple pluralities of pores, which in some cases are interconnected with each other, and in other cases are not interconnected with each other (although the pores within a plurality may still be internally interconnected).

According to some embodiments, the pores of the porous structure 26 are defined by one or more strands. The strands can include any suitable component or material capable of defining one or more pores, such as one or more meshes, grids, filaments, foams, lattices, wires, grates, strings, springs, coils, coil packs, webs, laces, filigrees, frames, frets, screens, reticulations, threads, tendrils, fibers, or other porous components or components that can define pores around them (whether they form strips, porous bodies, or other structures or configurations).

Where the porous structure 26 includes one or more strands, the strands can have any configuration suitable for forming the porous structure. For example, in some embodiments, the strands (or portions thereof) are straight, curved, spiraled, twisted, zig-zagged, accordioned, branching, randomly dispersed, geometrically arranged, interwoven, interconnected, separate, independent, uniform, layered, coupled with other strands, independent of one or more other strands, crossed with other strands, woven together, parallel to each other, diagonal to each other, placed with any suitable orientation with respect to each other, varied, or otherwise configured in any suitable manner that allows the implant 10 to function as described herein. In some embodiments, one or more of the strands are arranged in a spring-like configuration such that a stiffness is derived from compression of one or more of the strands. According to some embodiments, a combination of the material from which the porous structure is formed and the spring-like shape of the material results in a spring constant that yields the desired stiffness of the implant 10 (or the stiffness of one or more of the implant's load bearing regions).

In embodiments in which the porous structure 26 comprises one or more strands, the strands can also extend through any portion of the porous structure and in any orientation. For example, the strands (individually or collectively) can extend through the whole porous structure or through only a portion of the porous structure.

Where a strand includes a spring-like configuration, any shape can be used that results in spring-like or resilient action of the strand. For example, some embodiments of the strand include a configuration of a torsion spring, a coil spring, a conical spring, a compression spring, a helical spring, a leaf spring, a triangular spring, a rectangular spring, a diamond spring, a spring with another geometric configuration, a single-wire spring, a multi-wire spring, a spring grid, any other suitable type of spring, including as discussed in the PCT application publication WO2017100366A1 (entitled “Porous interbody spacer”) which is incorporated herein by reference in its entirety. Furthermore, multiple strands operating as springs can operate dependently of each other (e.g., they are interconnected or coupled in a series configuration) or independently of each other (e.g., they are not interconnected, or they are coupled in a parallel configuration). Even where strands are not interconnected (e.g., not physically joined directly together), strands can be interwoven with each other, such that one strand passes through a negative space not occupied by the other strand (but within the boundaries defined by such strand) without connecting to (or, in some cases, without even contacting) the other strand.

The porous structure 26 can have any number of strands, which can be part of one or more groups of strands having similar characteristics. For example, in some embodiments, the first strand is interconnected with one or more additional strands (which, in some cases, have a similar size, shape, stiffness, or other configuration as the first strand). Similarly, in some embodiments, the second strand is interconnected with one or more additional strands (which, in some cases, have a similar size, shape, stiffness, or other configuration as the second strand). Indeed, some embodiments have between 1 and 10,000 strands, or any subrange thereof. For example, some embodiments have between 10 and 5,000 strands, 20 and 500 strands, 50 and 200 strands, or any other number of strands.

Notwithstanding the foregoing, some embodiments of the body of the implant 10 do not include a porous structure 26. Rather, in some cases, the body is solid or semi-solid, rather than porous. Thus, the fixation mechanisms discussed herein can be used with a wide variety of implants, whether hollow, solid, porous, flexible, stiff, or otherwise configured. Thus, the porous structures described herein show an illustrative example of an implant body suitable for use in connection with the fixation mechanisms described in additional detail below.

According to some embodiments, the implant 10 includes one or more primary fixation mechanisms 28 configured to anchor the implant in a desirable location within a patient's spine. The primary fixation mechanism can include any component configured to assist in such fixation of the implant in its intended location.

Some embodiments of the primary fixation mechanism include one or more anchors 30 (which can be at least partially disposed within one or more anchor cavities 31), and actuators 32 (which can include one or more cams 34 or any other suitable features allowing the actuators to actuate the anchors).

According to some embodiments, the primary fixation mechanism 28 includes one or more anchors 30. The anchors can include any suitable components configured to fix the implant 10 to one or more bony endplates adjacent to the implant. For example, the anchors can include one or more blades, spikes, hooks, pins, shafts, plates, or other suitable anchoring components.

Where the anchors include blades, the blades can be any suitable type of blade. For example, in some cases, the blade is significantly wider and taller than it is thick (e.g., at least 175% (or any suitable amount more, such as at least 200%) wider and taller than it is thick). In some cases, the blade has an aspect ratio (width-height-thickness) of at least 3-3-1, or any other suitable ratio (e.g., 5-5-1, 10-10-1, 15-10-1, or another longer and taller than thick ratio). Indeed, in some cases, a thinner blade can cause less damage to the bony endplate (provided the blade is thick enough to withstand the stresses exerted upon it during the fulfillment of its function). Accordingly, in some cases, the blade is thin and comprises any suitable thickness of less than 3 mm thick at a portion of the blade that is configured to extend into a bony endplate (e.g., less than 2 mm thick, less than 1 mm thick, less than 0.5 mm thick, or any other suitable thickness). That said, some embodiments of the blade are wide enough to provide a significant anchor to the bone (e.g., at least 2 mm wide). Similarly, some embodiments of the blade are tall enough to insert a significant distance into the bony endplate to provide a strong anchor (e.g., at least 1 mm tall, or configured to insert at least 0.9 mm into the bony endplate). By way of non-limiting illustration, FIGS. 1-3, 4B-6B, and 6D-8B show anchors 30, including a cranial anchor 30a and a caudal anchor 30b, each of which includes a blade configured to insert into a bony endplate, the blade being significantly wider and taller than it is thick (the width of the anchors extending in a lateral direction with respect to the implant).

Where the anchor 30 includes one or more blades, the blade can also have any shape that allows it to insert into the bony endplate. For example, some embodiments are thinner (e.g., sharpened) toward an upper edge (in the case of a cranial anchor 30a) or a lower edge (in the case of a caudal anchor 30b), such that the sharpened edge is configured to enter the bony endplate while causing minimal damage. Similarly, some embodiments of the blade have a decreased width at the sharpened edge (e.g., the edge is tapered inward) to avoid unnecessary damage to the bone. While some embodiments of the blade are straight (e.g., across its width or its length), some embodiments also include a modified shape, such as one or more waves, curves, grooves, ridges, scallops, bends, curved portions, or any other suitable features that deviate from simply a straight line. Indeed, in some embodiments, the blade is generally straight, such as highly straight (e.g., within 1 degree of straight), substantially straight (e.g., within 3 degrees of straight), or largely straight (e.g., within 10 degrees of straight), or even somewhat straight (e.g., within 45 degrees of straight). Similarly, some embodiments of the anchor are generally planar, such as highly planar, substantially planar, or largely planar, although variations in 3 dimensional shape are included in some embodiments.

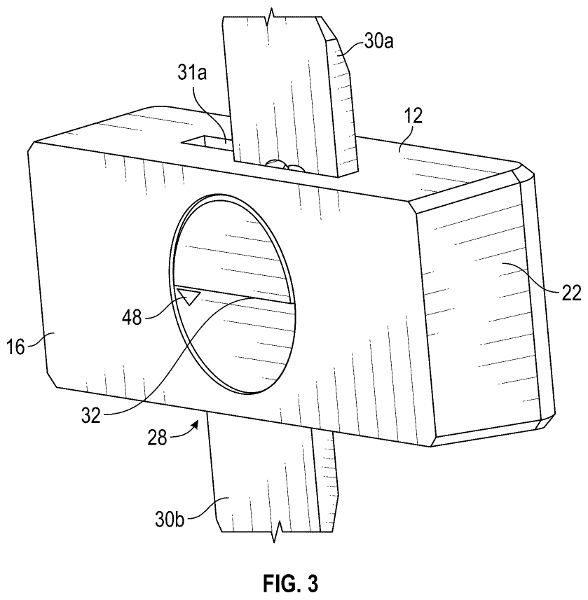

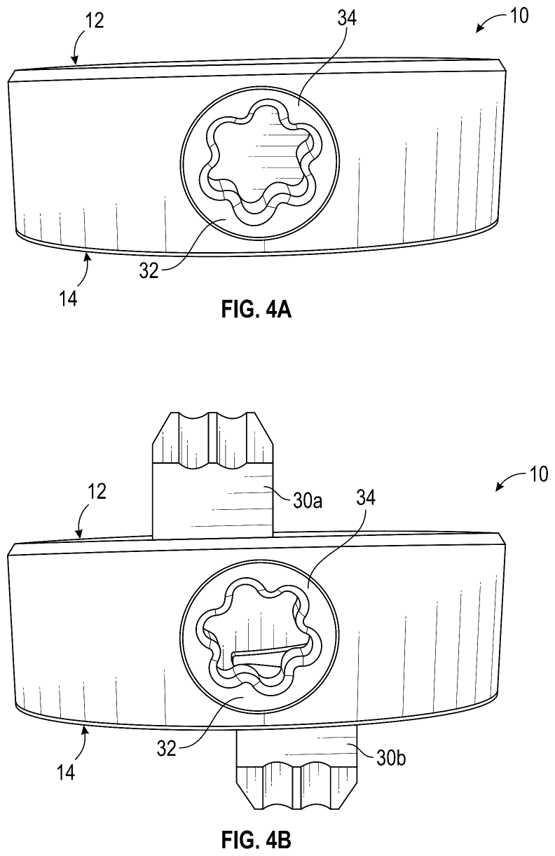

While the blades can have any suitable orientation, some embodiments of the blades are configured to be generally orthogonal, such as highly orthogonal (e.g., within 1 degree of orthogonal), substantially orthogonal (e.g., within 3 degrees of orthogonal), or largely orthogonal (e.g., within 10 degrees of orthogonal) to a direction of insertion into the applicable bony endplate. For example, in some embodiments in which the implant 10 is configured to be inserted from a ventral direction (e.g., where an inserter interface 23 is included on a ventral surface 16 of the implant), the blades are oriented highly orthogonal, substantially orthogonal, or largely orthogonal to the ventral surface. As another example, in some embodiments in which the implant 10 is configured to be inserted laterally (e.g., where an inserter interface 23 is included on a first lateral surface 20 or a second lateral surface 22), the blades are oriented highly orthogonal, substantially orthogonal, or largely orthogonal to the applicable lateral surface. In some cases, the orientation remains generally the same, such as highly the same (e.g., within 1 degree), substantially the same (e.g., within 3 degrees), or largely the same (e.g., within 10 degrees) while the blades transition from a deployed configuration to an undeployed configuration or vice versa (deployment of the blades is discussed in additional detail below). By way of non-limiting illustration, FIG. 4A shows an example of an implant 10 with blades in a retracted or undeployed configuration, while FIG. 4B shows an example of an implant 10 with blades 30a, 30b in an extended or deployed configuration. Although the blades are not well-seen in FIG. 4A due to being retracted into the body of the implant 10, the blades in each case (whether deployed or undeployed) are generally orthogonal to the cranial surface 12 and the caudal surface 14 of the implant 10.

In some embodiments, the anchors 30 are configured to be generally orthogonal (e.g., orthogonal, highly orthogonal, substantially orthogonal, or largely orthogonal) to at least one of the cranial surface 12 and the caudal surface 14. In some cases, the cranial anchor 30a is generally orthogonal to the cranial surface, and the caudal anchor 30b is generally orthogonal to the caudal surface. Moreover, in some embodiments, the anchors are configured to enter the patient's bony endplate generally orthogonal to the bony endplate. For example, while the anchors can extend from the implant body in any suitable manner, in some cases, the anchors are configured to be inserted into the vertebral body generally orthogonally, such as within: 1 degree of orthogonal, 3 degrees of orthogonal, 5 degrees of orthogonal, 10 degrees of orthogonal, 15 degrees of orthogonal, or at any other suitable angle within 45 degrees of orthogonal (more orthogonal than parallel) to the bony endplate. In some cases, orthogonal insertion is less likely (than some conventional devices) to break or split the bone, and more likely (than some conventional devices) to prevent the implant from migrating.

Where multiple anchors 30 are included (e.g., a cranial anchor 30a and a caudal anchor 30b, or any other anchor or additional anchor), the anchors can have any suitable positioning with respect to each other. For example, in some cases, the anchors are configured to align end-to-end (e.g., a base of one anchor has a same or similar ventral-dorsal position and lateral position as a base of another anchor, such that the anchors are significantly offset only in a cranial-caudal direction). That said, in some embodiments, the anchors are offset in a dorsal-ventral direction (e.g., one anchor is closer to the ventral surface 16 than is the other). In some embodiments, the anchors are offset in a lateral direction (e.g., one anchor is closer to a first lateral surface 20 than the other). This notwithstanding, in some embodiments, one or both (or more) of the anchors is configured to be disposed across a midsagittal plane (transecting the implant 10 into a first lateral half and a second lateral half). Thus, the anchors can be generally centrally disposed, even where they are offset from each other.

According to some embodiments, the implant 10 includes one or more anchor cavities 31. The anchor cavities can include any component suitable for housing one or more anchors 30. For example, the anchor cavities can include one or more depressions, slots, slits, recesses, grooves, receptacles, or other cavities formed in the implant body allowing the anchors to be at least partially or completely housed therein. In some cases, the anchor cavities include one or more cranial anchor cavities 31a (e.g., corresponding to one or more cranial anchors 30a), and in some cases, the anchor cavities include one or more caudal anchor cavities 31b (e.g., corresponding to one or more caudal anchors 30b). By way of non-limiting illustration, FIG. 5 shows an implant 10 having a cranial anchor cavity 31a out through which a cranial anchor 30a is configured to extend, as well as a caudal anchor cavity 31b out through which a caudal anchor 30b is configured to extend.

While some embodiments of the anchor cavities 31 are separate (e.g., the cranial anchor cavity 31a is separate and distinct from the caudal anchor cavity 13b), some embodiments of the anchor cavities are joined together (e.g., fluidically connected, sharing a common cavity space, or otherwise joined).

According to some embodiments, the anchors 30 are configured to be at least partially disposed within the anchor cavities 31 (e.g., the cranial anchor 30a is configured to be at least partially disposed within the cranial anchor cavity 31a, and the caudal anchor 30b is configured to be at least partially disposed within the caudal anchor cavity 31b) at least when the anchors are in an undeployed configuration. In some cases, the anchors are configured to be entirely disposed within the respective anchor cavities when the anchors are in the undeployed configuration. Furthermore, in some embodiments, the anchors are configured to be at least partially disposed within the respective anchor cavities when the anchors are in a deployed configuration (e.g., a base of each respective anchor remains within the anchor cavity, even where a leading edge (e.g., a blade portion) of the anchor is disposed outside of the cavity so that it can interface with the patient's bony endplate). By way of non-limiting illustration, FIG. 5 shows an implant 10 with a cranial anchor 30a and a caudal anchor 30b in an undeployed configuration, such that the cranial anchor 30a is fully disposed within a cranial anchor cavity 31a and the caudal anchor is fully disposed within a caudal anchor cavity 31b. By way of further illustration, FIGS. 2-3 show implants 10 whose anchors 30a, 30b are in a deployed configuration, with the bases of such anchors being seated within their respective anchor cavities.

According to some embodiments, the anchor cavities 31 have openings configured to allow the anchors 30 to be deployed therefrom. Although the openings can have any shape (e.g., slit-shaped (e.g., generally linear), circular, semi-circular, triangular, square, rectangular, trapezoidal, pentagonal, hexagonal, star-shaped, T-shaped, C-shaped, U-shaped, W-shaped, polygonal, symmetrical, asymmetrical, or any other regular or irregular shape), some embodiments of the openings have a shape corresponding to a shape of one or more of the anchors. In some embodiments, the opening of an anchor cavity (e.g., a cranial anchor cavity 31a or a caudal anchor cavity 31b) has a shape corresponding to a shape of both a cranial anchor 30a and a caudal anchor 30b, such that either anchor can be accessed through the opening. By way of non-limiting illustration, FIG. 5 shows a cranial anchor cavity 31a having an opening with a shape that has a first portion from which a cranial anchor 30a is configured to extend and a second portion (joined to but offset from the first portion) providing access to a base of the caudal anchor 30b.

According to some embodiments, the primary fixation mechanism 28 includes one or more actuators 32. The actuator can include any suitable component for deploying the anchors 30 (thereby shifting them from an undeployed position to a deployed position in which the anchors are configured to be affixed to the patient's bony endplates). For example, some embodiments of the actuator include automatic or semi-automatic components, such as one or more motors (e.g., stepper motors, servomotors, bushed or brushless motors, induction motors, or any other motors), hydraulic actuators, pneumatic actuators, electric actuators, linear actuators, piezoelectric actuators, thermal actuators, electromagnetic actuators, or other actuators configured to move the anchors. That said, some embodiments of the actuator include one or more manual mechanical components (e.g., configured to actuate the anchors in response to manual movements from a practitioner, such as a prying motion, lever action, manual rotation, twisting motion, pressing motion, pulling motion, sliding, or otherwise).

According to some embodiments, the actuator 32 is configured to output linear motion-such as highly linear (e.g., within 1 degree of linear), substantially linear (e.g., within 3 degrees of linear), largely linear (e.g., within 10 degrees of linear) motion, or other generally linear motion. For example, some embodiments are configured to shift the anchors 30 linearly from an undeployed position (e.g., within the respective anchor cavities 31 within the implant body) to a deployed position (e.g., at least partially external to the anchor cavities such that they penetrate the bony endplates of vertebral bodies adjacent to the implant). Accordingly, deployment of the anchors in some embodiments involves translational movement where the anchors move straight (or highly, substantially, or largely straight) into the bony endplate without rotating, twisting, sliding, or otherwise being moved into place in a manner that could cause additional damage to the endplate.

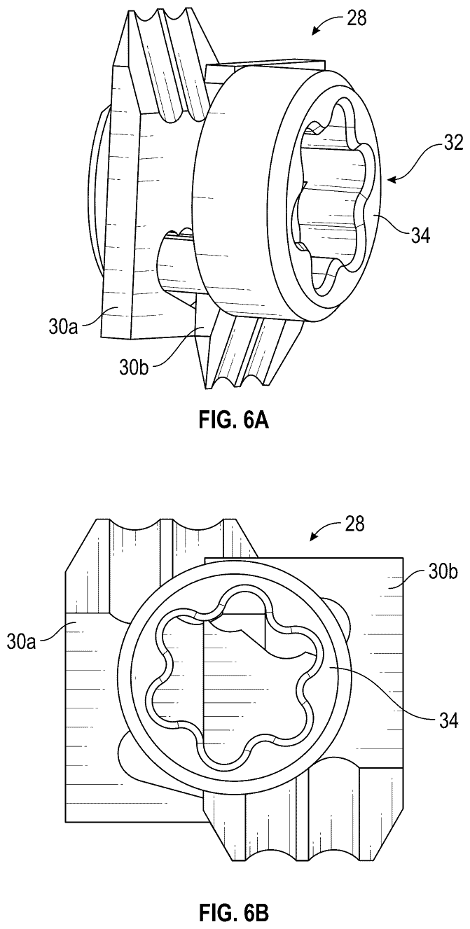

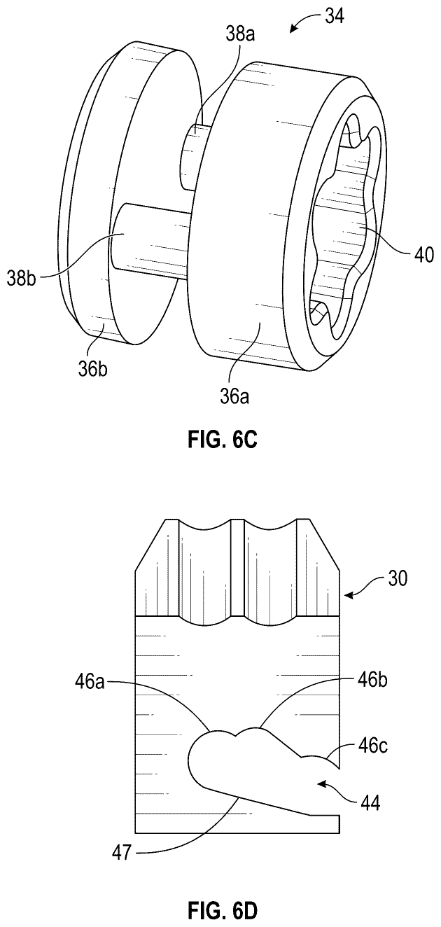

In some embodiments, despite outputting linear motion, the actuator 32 is configured to input a different type of motion (although some embodiments also input linear motion). Some embodiments are configured to receive electrical input, linear mechanical input (e.g., in a same or a different direction as the output), chemical input (e.g., a reaction causing a material to expand and force the anchors away from each other), hydraulic input, rotational mechanical input, translational input, or any other suitable kind of input. For example, in some embodiments, the actuator is configured to receive rotational motion, subsequently converting that rotational motion into translational motion, as described above. By way of non-limiting illustration, FIG. 5 shows an implant 10 having a primary fixation mechanism 28 in which an actuator 32 is configured to receive rotational mechanical motion as an input, and to output linear (translational) mechanical motion to deploy the anchors 30a, 30b.

Where the actuator 32 is configured to convert rotational input to translational output, the actuator can do so in any suitable manner. According to some embodiments, the actuator includes one or more cams 34. Where a cam is included, the cam can have any suitable configuration or components that enable it to convert rotational motion into translational motion. For example, the cam can include one or more shafts, drive gears, bearings, lobes, catches, or any other features having any suitable pitch points, pitch curves, pressure angles, trace points, pitch circles, base circles, or any other suitable cam geometry as may be useful to provide the functions discussed herein. For example, some embodiments of the cam include one or more lobes 36 (e.g., a first lobe 36a and a second lobe 36b, along with any suitable number of additional lobes) joined together by one or more shafts 38 (e.g., a first shaft 38a and a second shaft 38b, along with any suitable number of additional shafts). By way of non-limiting illustration, FIG. 6C shows a cam 34 having a first lobe 36a and a second lobe 36b separated (and coupled together) via a first shaft 38a and a second shaft 38b.

Where the cam 34 includes one or more lobes 36, the lobe can have any shape allowing it to rotate (e.g., having a circular, semi-circular, triangular, square, rectangular, trapezoidal, pentagonal, hexagonal, star-shaped, T-shaped, polygonal, elliptical, symmetrical, asymmetrical, or any other regular or irregular shaped cross-section). In some cases, the lobe is shaped to directly convert rotational motion to translational motion, whereas in some cases, another component of the cam (such as a shaft 38) is configured to convert the motion. In some cases, the lobe is generally cylindrical (e.g., with a circular or oval cross section), for smooth rotation. For example, some embodiments of the lobe resemble a flat cylinder (e.g., pancake shaped).

Where the cam 34 includes multiple lobes 36, the lobes can have any suitable position with respect to each other. For example, although the lobes may be offset in some cases, some embodiments of the lobes are generally aligned (e.g., laterally and caudal-cranially) such that they overlap (e.g., highly, substantially, or largely) in a dorsal-ventral direction.

The lobes 34 of some embodiments are separated by a distance (e.g., via the shaft 38). While the distance can be any suitable distance, in some cases the distance is approximately equal to a thickness of the anchors 30 (±1%, 2%, 3%, 5%, or 10%, in some cases), such that at least a portion of the anchors can fit between the lobes. In some cases, the lobes thus help to hold the anchors in place, or in a proper orientation (e.g., substantially parallel to the ventral surface 16, or substantially orthogonal to the cranial surface 12 or the caudal surface 14, or another orientation as discussed herein).

Where the cam 34 includes one or more shafts 38, the shaft can have any shape that allows it to couple the first lobe 36a to the second lobe 36b or to assist in the translational movement of one or more anchors 30. For example, the shaft can include one or more dowels, rods, columns, hooks, sheets, pawls, catches, engagements, fasteners, or other any other suitable coupling components. While the shaft can have any suitable position with respect to the lobe, some embodiments of the shaft are offset from a center of the lobe such that the shaft travels in a cranial-caudal direction when the lobe is rotated. In some cases, the shaft is disposed at or near a perimeter of the lobe. Accordingly, in some embodiments, the shaft is configured to press up (cranially) or down (caudally) on an anchor to cause the anchor to move cranially or caudally (as applicable) when the lobe rotates. Where multiple shafts are included, the shafts can be configured to each act on a different anchor, or (in some cases) multiple shafts can be configured to act in concert on a single anchor. By way of non-limiting illustration, FIG. 6C shows a cam 34 having a first lobe 36a separated from a second lobe 36b by a first shaft 38a and a second shaft 38b, the lobes being substantially aligned, the shafts being disposed near a perimeter of the lobes, thereby coupling the lobes together and being configured to have a cranial-caudal component of movement (in addition to a lateral component) as the lobes rotate around a central axis of the cam.

According to some embodiments, the cam 34 includes one or more engagement features 40. The engagement feature 40 can include any suitable feature allowing a tool to engage with a rotate the cam, such as one or more drives (or drive recesses), slots, grooves, teeth, hexes, magnetic interfaces, processes, recesses, or any other features configured to engage with a corresponding key 42 (or other tool or part thereof) to allow for rotation of the cam. By way of non-limiting illustration, FIG. 6C shows a cam 34 having an engagement feature 40 in the form of a recess having a general hex shape, such that the cam can be engaged and rotated using a key with a process having a corresponding hex shape.

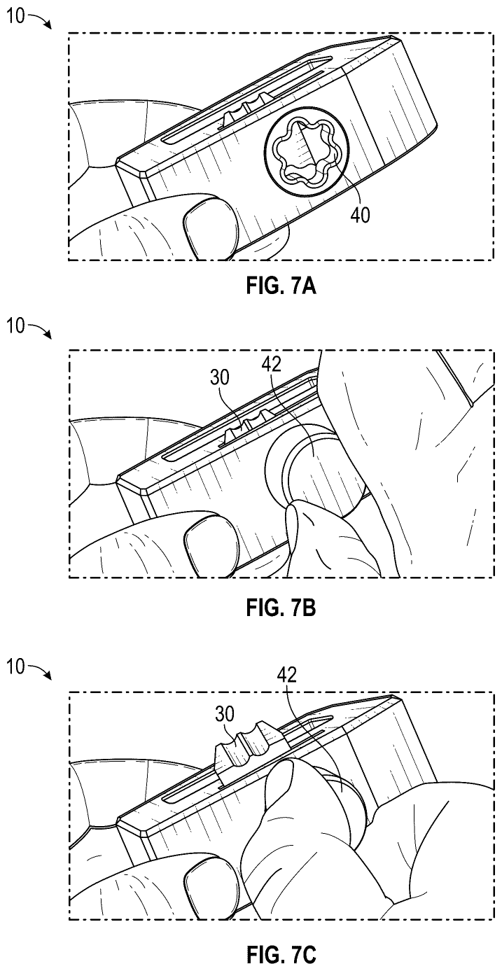

In accordance with the foregoing, some embodiments include one or more keys 42 configured to engage with the engagement feature 40. The key can include any component for interfacing with the engagement feature, thereby allowing rotation of the cam 34 and actuation of the anchors 30. In some cases, the key is provided as a stand-alone tool, but in some cases, the key is provided as part of another tool (e.g., a driver, a deployment tool, an insertion tool, or any other suitable tool). In some cases, the key is provided as part of an insertion tool, such as an insertion tool configured to interface with an inserter interface 23 as discussed above. In such cases, the key can be included with any part of the insertion tool, such as on an end of an expansion shaft of an interbody inserter (e.g., thereby enabling the expansion shaft to actuate the anchors via rotation in addition to locking flexible tabs and preventing dissociation of the interbody inserter from the implant 10). By way of non-limiting illustration, FIGS. 7A-7C show an implant 10 with anchors 30 being translationally actuated as a result of engagement of a key 42 with an engagement feature 40 and rotation of the key.

The anchor 30 itself can have any configuration that allows it to be translationally actuated by the cam 34. That said, some embodiments of the anchor include one or more specific features, such as one or more catches, engagements, recesses, grooves, protrusions, or other interfaces (also referred to as cutouts) 44, configured to interface with one or more parts of the cam for smooth or efficient actuation.

Where the anchor includes a cutout 44, the cutout can be any shape that allows the anchor to be slid onto or otherwise disposed overlapping one or more shafts 38 of the cam 34. Thus, in some cases, the cutout allows the shaft to be disposed in an interior of the anchor (between a first lateral side of the anchor and a second lateral side of the anchor, as well as between a leading edge (e.g., a cranial edge in the case of a cranial anchor 30a, or a caudal edge in the case of a caudal anchor 30b) and a base (or a trailing edge, disposed opposite the leading edge). In some cases, the cutout allows the anchor to be actuated from an interior portion of the anchor, thus minimizing the likelihood of the anchor rotating, shifting, bending, or otherwise deviating from its desired path as it is pressed into the bony endplate. Further, in some cases, the cutout acts to help prevent the anchor from moving too far into the bony endplate, as the base or trailing edge of the anchor is prevented (by the shaft) from moving farther in the direction of the anchor's insertion.

According to some embodiments, the cutout 44 includes one or more contours 46 configured to affect the interaction between the cutout and the shaft 38. While the contours can be configured to affect such an interaction in any suitable manner, some embodiments of the contours are configured to do one or more of the following: provide a smoother translational motion as a result of the rotational motion of the cam; provide a multi-tiered translational motion, thereby allowing a practitioner to receive tactile feedback (or visual feedback or any other type of feedback) indicating how far an anchor has been inserted into the bony endplate; provide a number of pre-set anchor retention heights; prevent retrograde (e.g., in a direction opposite the insertion direction) movement of the anchor (e.g., by unidirectionally locking rotation of the cam); allow the shaft to remain closer to the center of the anchor as the shaft rotates; enable the shaft to exert force on the anchor primarily in the desired direction (e.g., substantially orthogonal to the cranial surface 12 or caudal surface 14, as applicable) even as the lateral position of the shaft changes due to its rotation; allow for easier entry of the anchor into the bony endplate; modulate the amount of force exerted on the anchor at different points along its translational path; or otherwise affect the translational path of the anchor as generated from the rotational movement of the cam. By way of non-limiting illustration, FIG. 6D shows an anchor 30 having a cutout 44 with a plurality of contours 46, including a first contour 46a, a second contour 46b that extends slightly farther toward a leading edge of the anchor than the first contour, and a third contour 46c disposed adjacent to a lateral edge of the anchor, and which is less prominent than the first contour and the second contour (e.g., it does not extend as far toward the leading edge).

Some embodiments of the cutout 44 include one or more retraction surfaces 47 (e.g., opposite the contours 46). The retraction surface can have any suitable feature allowing it to enable retraction of the anchor 30 (e.g., by backward motion of the shaft 38 pushing against the retraction surface, thereby forcing the anchor back into the implant body). For example, some embodiments of the retraction surface have contours (including any of the features of contours discussed above). By way of non-limiting illustration, FIG. 6D shows an anchor 30 having a retraction surface 47 that is sloped toward a base of the anchor and toward a lateral opening of the cutout 44.

Although some embodiments of the primary fixation mechanism 28 are configured to deploy multiple anchors 30 (e.g., the cranial anchor 30a and the caudal anchor 30b) simultaneously, some embodiments of the anchors are configured to be sequentially deployed. In some embodiments, the anchors are configured to be either simultaneously or selectively deployed, with the practitioner being able to choose which anchor to deploy and how much to deploy it or being able to choose to deploy both anchors together.

In some embodiments, the anchors 30 are configured to be self-retaining. For example, in some embodiments, the anchors are barbed or otherwise configured not to retract from the bone. In some cases, the anchors are configured to be retained through the use of one or more additional mechanisms. For example, in some cases, the implant 10 is configured to have one or more local minima in potential energy when the anchors are in the deployed configuration (e.g., through the use of one or more detents to help prevent the anchor from collapsing back into the implant). Thus, in some cases, in order for the anchor to retract from the deployed configuration to the undeployed configuration, it must first move to a position of higher potential energy before shifting back to a lower energy state. To illustrate, in some embodiments, the primary fixation mechanism 28 includes one or more interference fits between the anchor 30 and the corresponding anchor cavity 31. In some embodiments, the primary fixation mechanism includes one or more stops (e.g., objects configured to be inserted between an anchor and a portion of the implant body to prevent the anchor from retracting back into the implant). In some embodiments having one or more stops, the stops are configured to be disposed between two anchors, thereby preventing them from moving in a retrograde direction.

Notwithstanding the foregoing, in some embodiments, the anchors are retained due to a stop, lock, or limiter (as discussed in more detail below) in the primary fixation mechanism 28, such that the anchors can be selectively retracted if desired. Accordingly, in some embodiments, the anchors 30 are configured to be selectively retracted (e.g., so as to be partially or completely retracted into a body of the implant 10). Even where the anchors are selectively retractable, some embodiments of the anchors are configured to resist being inadvertently pushed back into the body of the implant (e.g., force exerted on the anchors does not result in retrograde movement). However, some embodiments include a disengagement mechanism (a key 42, a lever, a pawl, or any other suitable instrument or mechanism), which can be used to selectively retract one or more of the anchors (e.g., by rotating the cam 34 in a direction opposite the direction for deployment).

According to some embodiments, the actuator 32 includes one or more limiters 48. The limiter can include any component configured to limit (e.g., slow, increase the force required to accomplish, prevent, or otherwise limit) any particular motion of the actuator (in some cases, selectively). For example, in some cases, the limiter limits retrograde motion of the anchors 30 (e.g., preventing or impeding their unintentional retraction from the bony endplate). The limiter can also limit retrograde rotation of the cam 34 (e.g., where the cam is configured to rotate counterclockwise to deploy the anchors, the limiter can prevent the cam from rotating counterclockwise), lateral motion of the shaft 38, or any other motion that is undesired or that is desired only under certain circumstances.

In some embodiments, the limiter 48 includes one or more over-rotation slots (or any other suitable feature) to prevent disassociation of the anchors. For example, in some cases, during assembly, the anchors are pressed into place. In some such cases, when rotated in the direction of deployment (e.g., clockwise), one or more over-rotation slots, divots, detents, or other features pop into place to prevent retrograde rotation to the retracted position. By way of non-limiting illustration, FIGS. 8A-8B show some embodiments of implants 10 having limiters 48 that include over-rotation divots or over-rotation bumps configured to prevent over-rotation of the cam 34.

According to some embodiments, the limiter 48 is configured to prevent a force exerted on the anchors 30 from causing the anchors to retract (e.g., a force pushing on the leading edges of the anchors), while still allowing a rotational force exerted on the cam 34 to retract the anchors. In some cases, this is accomplished in concert with a functionality of the cam. For example, in some embodiments, the cam is configured to rotate, and the shafts are configured to move (e.g., in a clockwise rotation) such that they shift from a lateral position to a cranial or caudal position in response to the rotation, thereby deploying the anchors (forcing them out of the respective anchor cavities 31 and into the bony endplates of the patient's spinal segments). Once the shafts crest the cranial-most or caudal-most positions, the anchors are fully deployed, and the shafts would normally begin to descend on the other side of the cam as the cam continued its rotation. However, in some cases, the limiter includes a pin, a stop, or another component configured to prevent continued rotation substantially past the apex. This notwithstanding, in some cases, the cam is configured to rotate slightly past (e.g., anywhere between 1 degree past and 90 degrees past, or any subrange thereof, such as between any of 1 degree past, 2 degrees past, 3 degrees past, 5 degrees past, 10 degrees past, 15 degrees past, 30 degrees past, and 45 degrees past) the point where the shafts crest the cranial-most and caudal-most positions, such that the shafts have begun their downward portion of the rotation before being stopped by the limiter. Accordingly, when a force is applied to the anchors, the shafts will naturally be inclined to continue in their path of rotation (as opposed to being inclined to counter-rotate and again crest the cranial-most or caudal-most point), only to be barred from such rotation by the limited. Thus, in such cases, an increase in potential energy is required to undeploy the anchors, as the cam must be counter-rotated, causing the shafts to again crest the peaks of highest energy. By way of non-limiting illustration, FIGS. 9D-9E show a cross section of an implant 10 (in particular, cross section A-A of FIG. 9C) highlighting a primary fixation mechanism 28. FIG. 9D shows a position in which a cam has not yet rotated to an apex, such that shafts 38a, 38b have not yet reached their cranial-most and caudal-most positions respectively (e.g., the first shaft 38a is in an “11:59” clock position as part of a clockwise rotation), and the anchors 30a, 30b are not yet fully deployed. In this position, a force applied to the leading edges of the anchors would cause the shafts to rotate counter-clockwise, thereby causing the anchors to undeploy back into the body of the implant. However, FIG. 9E shows a position in which the anchors 30a, 30b are fully deployed, as the cam has rotated to the point where the shafts have crested the cranial-most and caudal-most positions (e.g., the first shaft 38a is in a “12:01” clock position as part of the clockwise rotation). In this position, a force applied to the leading edges of the anchors would cause the shafts to continue with their clockwise rotation (as energy would be required to instead reverse the direction of the rotation and instead again crest the energy apex). However, further clockwise rotation is eventually barred by the limiter 48, which can be placed in any suitable position to ensure that the anchors 30 are deployed to a desirable height, and that sufficient energy would be required to reverse the direction of rotation so as to ensure that accidental undeployment due to forces encountered in typical physiological applications is not possible (yet where selective undeployment is still possible by using a key and manually counter-rotating the cam).

According to some embodiments, one or more surfaces or components of the implant 10 are configured to be flush with the body of the implant (e.g., one or more surfaces of the implant) thereby avoiding protrusions or indentations that could get caught on part of a patient or accumulate matter. In some cases, one or more plugs (or plugging materials) are provided to fill one or more cavities in the implant. Indeed, in some embodiments, the primary fixation mechanism 28 is configured to receive a plug.

Where any of the components herein are described as being coupled together (or coupled to another component), such coupling can take any form suitable for selectively, permanently, or semi-permanently coupling such components together as appropriate. For example, coupling can include: coupling via couplers such as nails, screws, bolts, staples, eyelets, magnets, hook-and-loop fasteners, interference fits, friction fits, mechanical engagements, tongue-and-groove connections, snaps, ties, rivets, or any other suitable coupling components or methods; integrally forming parts together (e.g., via additive manufacturing or any other suitable method to ensure that the components form a single cohesive (e.g., monolithic) unit); welding; use of an adhesive; or any other suitable coupling mechanism.

The systems and methods described herein can be modified in any suitable manner. For example, in some cases, the implant 10 includes not only a primary fixation mechanism 28, but also a secondary fixation mechanism. Where a secondary fixation mechanism is included, the secondary fixation mechanism can include any of the features of the primary fixation mechanism (e.g., an additional actuator formed in the same or a different surface, additional anchors for redundancy, etc.). Additionally, some embodiments of the secondary fixation mechanism include additional anchors (e.g., spikes, blades, or any other type of anchor as discussed herein) formed in different parts of the implant, such as along lateral portions of the cranial or caudal surfaces.

As another example of a suitable modification, in some cases, all or part of the actuator 32 is included within an insertion tool (as opposed to or in addition to being included within a body of the implant). That said, one potential downside to including a rotary to linear mechanism inside or with an insertion tool is that sometimes a force that is applied to deploy the anchors through the insertion tool can cause bending at the tip of the insertion tool, thereby causing the implant to disengage from the insertion tool. By integrating the lifting mechanism (the actuator) into the implant and transmitting a torque through the actuator, accidental disengagement of the insertion tool from the inserter interface 23 can be prevented. That said, some embodiments of the insertion tool include a reinforced attachment interface configured to strongly couple with the inserter interface so as not to disengage due to application of torque.

As another example of a suitable modification, in some embodiments, the described implant 10 is modified such that the primary fixation mechanism 28 is configured to deploy one or more anchors from any other suitable portion of the implant (e.g., one or more lateral sides, dorsal sides, or any other suitable portion of the implant).

Embodiments of the implant 10 can have any suitable feature. Indeed, in some cases, the features of the implant enable insertion and primary fixation in a manner that causes less damage to the patient's bony endplates and less pain to the patient overall. Furthermore, some embodiments of the implant are easier to insert or easier to deploy, thereby resulting in less time required for a practitioner to perform the operation, and reduced hard and intangible costs for the patient.

Any and all of the components in the figures, embodiments, implementations, instances, cases, methods, applications, iterations, and other parts of this disclosure can be combined in any suitable manner. Additionally, any component can be removed, separated from other components, modified with or without modification of like components, mixed, or otherwise altered together or separately from anything else disclosed herein.

As used herein, the singular forms “a”, “an”, “the” and other singular references include plural referents, and plural references include the singular, unless the context clearly dictates otherwise. For example, reference to an anchor includes reference to one or more anchors, and reference to detents includes reference to one or more detents. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

1. An interbody implant comprising:
an implant body comprising:
a cranial surface; and
a caudal surface disposed opposite the cranial surface; and
a primary fixation mechanism comprising:
an anchor cavity defined by the implant body;
an anchor configured to be housed at least partially within the anchor cavity in an undeployed position, wherein the anchor is configured to be translationally deployed to a deployed position in order to couple the interbody implant to a bony endplate of a spinal vertebra; and
an actuator configured to effectuate selective deployment of the anchor.

2. The interbody implant of claim 1, wherein the anchor comprises a blade.

3. The interbody implant of claim 2, wherein the blade is oriented substantially orthogonal to at least one of the cranial surface and the caudal surface.

4. The interbody implant of claim 3, wherein the blade is also oriented substantially parallel to a ventral surface of the interbody implant.

5. The interbody implant of claim 1, wherein the actuator comprises a cam configured to convert rotational motion of the cam to translational motion of the anchor.

6. The interbody implant of claim 5, wherein the cam comprises an engagement feature configured to interface with a key.

7. The interbody implant of claim 5, wherein the cam comprises a first lobe and a second lobe separated and coupled together by a shaft, wherein the shaft is configured to push on the anchor when the cam rotates to thereby actuate the anchor.

8. The interbody implant of claim 1, wherein the actuator comprises a limiter configured to prevent unintentional retraction of the anchor back into the anchor cavity.

9. The interbody implant of claim 1, further comprising an inserter interface configured to interface with an insertion tool.

10. An interbody implant comprising:
a cranial surface;
a caudal surface;
an implant body extending between the cranial surface and the caudal surface;
a first anchor cavity formed in the implant body and opening from at least one of the cranial surface and the caudal surface;
a first anchor at least partially disposed within the first anchor cavity; and
an actuator configured to convert rotational motion to translational motion to thereby deploy the first anchor translationally to a position at least partially exterior to the first anchor cavity.

11. The interbody implant of claim 10, wherein an orientation of the first anchor in an undeployed position is substantially the same as an orientation of the first anchor in a deployed position.

12. The interbody implant of claim 10, wherein a surface of the interbody implant comprises an inserter interface, and wherein the first anchor comprises a first blade oriented largely orthogonal to the surface comprising the inserter interface.

13. The interbody implant of claim 12, wherein the first blade is further oriented largely orthogonal to each of the cranial surface and the caudal surface.

14. The interbody implant of claim 10, further comprising a second anchor cavity formed in the implant body and opening from another of the at least one of the cranial surface and the caudal surface, a second anchor being at least partially disposed within the second anchor cavity, wherein the actuator is further configured to deploy the second anchor translationally to a position at least partially exterior to the second anchor cavity.

15. The interbody implant of claim 14, wherein when the first anchor and the second anchor are each in an undeployed position, the first anchor is disposed closer to a dorsal surface of the interbody implant and closer to a first lateral surface of the interbody implant than the second anchor.

16. The interbody implant of claim 14, wherein each of the first anchor and the second anchor is disposed across a midsagittal plane of the interbody implant.

17. An interbody implant system comprising:
an interbody implant comprising:
an implant body;
an inserter interface coupled to the implant body; and
a primary fixation mechanism comprising:
an anchor; and
an actuator configured to translationally deploy the anchor; and
an interbody insertion tool configured to selectively couple to the inserter interface, the interbody insertion tool comprising a key configured to activate the actuator.

18. The interbody implant system of claim 17, wherein the actuator comprises a cam having an engagement feature, wherein the key is configured to interface with the engagement feature.

19. The interbody implant system of claim 18, wherein a rotation of the cam using the key leads to a translational deployment of the anchor.

20. The interbody implant system of claim 17, wherein the interbody implant is configured to have a direction of insertion based on a location of the inserter interface, and wherein the anchor is oriented substantially orthogonal to the direction of insertion.
